(19) United States
(12) Patent Application Publication
(10) Pub. No.: US 2020/0020441 A1
(43) Pub. Date: Jan. 16, 2020

(54) SEAMLESS INTERFACING OF LABORATORY INSTRUMENTS

(71) Applicant: Beckman Coulter, Inc., Brea, CA (US)

(72) Inventor: Santosh V. Vijay, Placentia, CA (US)

(21) Appl. No.: 16/036,315

(22) Filed: Jul. 16, 2018

(51) Int. Cl. G16H 40/40; G16H 10/40; G05B 15/02; B65G 43/08

(52) U.S. Cl. CPC G16H 40/40; B65G 43/08; G05B 15/02; G16H 10/40

(57) ABSTRACT

Laboratory instruments may be interfaced into an existing laboratory subsystem. The subsystem may be obtained including a first instrument, a transport assembly, and a controller configured to communicate with the first instrument and the transport assembly using a first data format. A second instrument may be attached to the subsystem using an adapter device to form a completed sample processing system. The second instrument may communicate using a second data format. The completed sample processing system may include a translation module configured to convert data from the second data format to the first data format. The completed sample processing system may be used to process biological or chemical samples using the transport assembly, the first instrument, and the second instrument. The translation module may convert the data communicated from the second instrument to the subsystem by converting the data from the second data format to the first data format.

### BACKGROUND

Biological specimen testing may be used for identifying health issues of a patient, research and testing for pharmaceuticals, and many other reasons. Biological specimen testing often takes place in a laboratory with advanced equipment and instruments. In such a laboratory, various instruments may work together, connected through a transport assembly for moving the biological specimen from one instrument to another for completing a battery of tests. Coupling these instruments together into a laboratory system can be challenging. For example, when a new instrument is added to the laboratory system, the laboratory system may be required to undergo new approval from regulatory bodies, which is costly in both time and money. Because the entire laboratory system has changed, the entire laboratory system may be required to undergo the approval process. Further, in some cases, the transport assembly for connecting instruments together into a subsystem may be manufactured by a first company, but one or more of the instruments may be manufactured by a second company. Because there is not currently a universal standard used by manufacturers of laboratory system components (e.g., instruments, transport assembly, and so forth) for the physical or software connections, and because there is not a universal standard for the communication protocols, the instrument from the second company may not be compatible with the transport assembly manufactured by the first company. Because of the incompatibilities, the laboratory is either forced to use a single manufacturer for the entire laboratory or for example forego some of the automation capabilities available when a compatible transport assembly and instruments are used by having disparate instruments throughout the laboratory.

### BRIEF SUMMARY

A system of one or more computers can be configured to perform particular operations or actions by virtue of having software, firmware, hardware, or a combination of them installed on the system that in operation causes or cause the system to perform the specific actions on the system as a whole or on a part of the system. One or more computer programs can be configured to perform particular operations or actions by virtue of including instructions that, when executed by data processing apparatus, cause the apparatus to perform the actions on the system as a whole or on a part of the system. One general aspect includes a method for interfacing laboratory instruments to a subsystem. The method may include obtaining a subsystem. The subsystem may include a first instrument, a transport assembly, and a controller configured to communicate with the first instrument and the transport assembly using a first data format. The method may also include attaching a second instrument to the subsystem using an adapter device to form a completed sample processing system. The second instrument may communicate using a second data format. The completed sample processing system may include a translation module configured to convert data from the second data format to the first data format. The method may also include operating the completed sample processing system to process biological or chemical samples using the transport assembly, the first instrument, and the second instrument. The translation module may convert the data communicated from the second instrument to the subsystem from the second data format to the first data format.

Other embodiments may include corresponding computer systems, apparatus, and computer programs recorded on one or more computer storage devices, each configured to perform the actions of the methods.

Implementations may include one or more of the following features. In some embodiments, the adapter device is a wireless connection device that facilitates wireless communication between the second instrument and the transport assembly. In some embodiments, the adapter device includes a physical connection port for physically coupling the second instrument to the transport assembly.

In some embodiments, the translation module is installed in the controller. In some embodiments, the translation module is installed in the adapter device.

Implementations of the described techniques may include hardware, a method or process, or computer software on a computer-accessible medium.

### BRIEF DESCRIPTION OF THE DRAWINGS

A further understanding of the nature and advantages of various examples may be realized by reference to the following figures.

FIG. 1 illustrates a simplified block diagram of a completed sample processing system, according to an embodiment.

FIG. 2 illustrates a simplified block diagram of a completed sample processing system including software components, according to an embodiment.

FIG. 3 illustrates a flowchart depicting a method for creating a completed same processing system, according to an embodiment.

FIG. 4 illustrates a schematic diagram depicting an example computer system, according to an embodiment.

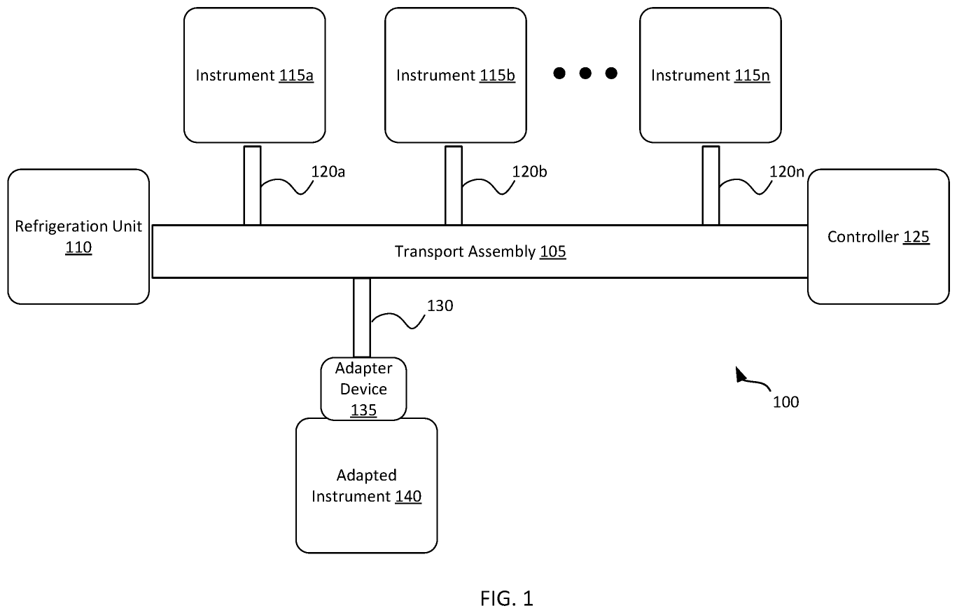

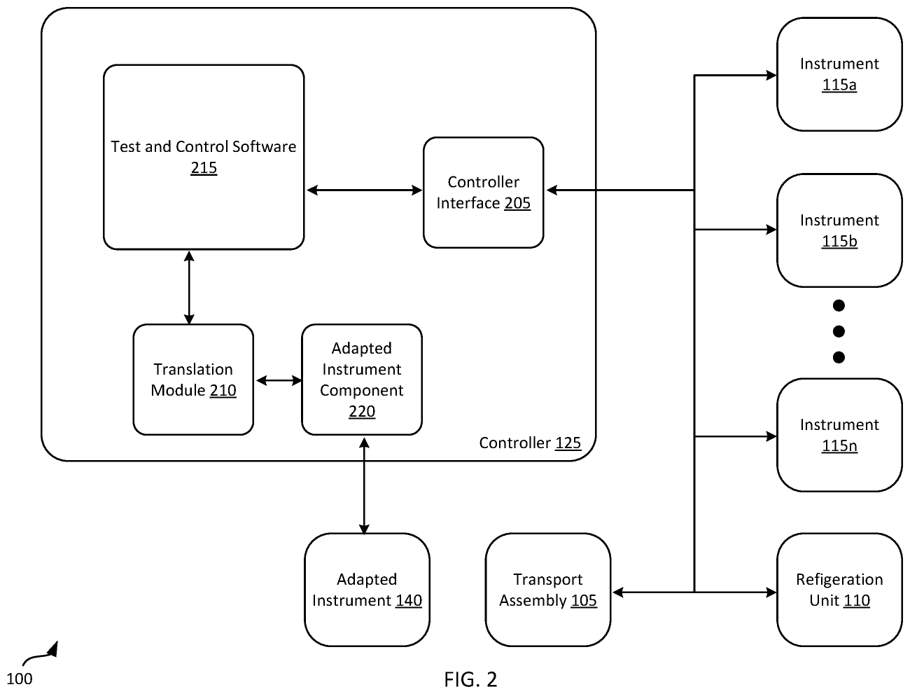

Unless otherwise indicated, elements using the same indicator number are the same elements between differing figures. For example, controller 125 in FIG. 1 is the same controller 125 depicted in FIG. 2. Some elements may include multiple of the same elements, which are indicated by a letter following the indicator number. For example, there may be any number of instrument 115, which is indicated by instrument 115a through instrument 115n.

### DETAILED DESCRIPTION

Embodiments include systems and methods for incorporating an instrument (e.g., a third-party instrument) into an existing laboratory subsystem. The laboratory subsystem can include a transport assembly and one or more instruments controlled by a controller. A second instrument may use a different data format than the laboratory subsystem. The second instrument may be coupled to the laboratory subsystem using an adapter device. The adapter device may physically or wirelessly couple the second instrument to the laboratory subsystem. Further, a translation module may translate data from the format used by the laboratory subsystem to the format used by the second instrument and vice versa.

Embodiments of the invention may be used to detect the presence, absence, or concentration of an analyte in a biological or chemical sample. Biological samples such as biological fluids may include, but are not limited to, blood, plasma, serum, or other bodily fluids or excretions, such as but not limited to saliva, urine, cerebrospinal fluid, lacrimal fluid, perspiration, gastrointestinal fluid, amniotic fluid, mucosal fluid, pleural fluid, sebaceous oil, exhaled breath, and the like. Chemical samples may include any suitable types of samples including chemicals including water samples.

Prior to discussing embodiments of the invention, it may be helpful to discuss some terms.

An “adapter device” may be a device that can allow one apparatus (e.g., instrument) to function with another apparatus (e.g., a transport assembly). In some embodiments, an adapter may be a hardware and/or a software component that facilitates coupling the second instrument (e.g., the third party instrument) to the subsystem and/or the transport assembly.

A “completed sample processing system” can be a processing system that includes an intended number of components, such that the processing system is capable of processing samples as intended. In some embodiments, a “completed sample processing system” can be a biological or chemical processing system that includes components including at least a subsystem (i.e., a controller, a transport assembly, and at least one instrument, where each component can communicate using a first data format), at least a second instrument that communicates using a second data format, which is different than the first data format, and a translation module for converting communications between the second instrument and the subsystem from the first data format to the second data format, and vice versa.

A “controller” may include hardware and/or software that can manage or direct the flow of data between two entities, and control the operation of one or more of the entities. In some embodiments, a “controller” can be a computer system that includes software applications that provides instructions to the subsystem and the completed sample processing system for moving samples (e.g., biological and/or chemical samples that are within test tubes/sample containers) from one instrument or location to another instrument or location.

A “first data format” may be a specific data format that is distinct from other data formats such as a second data format. In some embodiments, a first data format may be a data format for communicating within the subsystem and the completed sample processing system. The first data format is used by a controller to send instructions to a transport assembly and the instruments of the subsystem, and the completed sample processing system. The instructions may include, for example, instructions to move the test tubes/sample containers containing the biological and/or chemical samples from one instrument or location to a different instrument or location. The first data format may specify the type, order, length, and structure of data messages, including header information, used by the subsystem and within the completed sample processing system. In some embodiments, the first data format is a proprietary data format loped by the manufacturer of the transport assembly and instruments of the subsystem to be used within the subsystem.

An “instrument” may be any machine or equipment that performs a specified function or test. Examples of instruments include a recapper, capper, centrifuge, or aliquoter. Instruments may be coupled to the transport assembly and communicate with the controller.

A “memory device” may be any suitable device that can store electronic data. A suitable memory device may comprise a computer readable medium that stores instructions that can be executed by a processor to implement a desired method. Examples of memory devices may comprise one or more memory chips, disk drives, etc. Such memory devices may operate using any suitable electrical, optical, and/or magnetic mode of operation.

A “processor” may refer to any suitable data computation device or devices. A processor may comprise one or more microprocessors working together to accomplish a desired function. A processor can include a single-core processor, a plurality of single-core processors, a multi-core processor, a plurality of multi-core processors, or any other suitable combination of hardware configured to perform arithmetical, logical, and/or input/output operations of a computing device.

A “second data format” may be a data format that is distinct from other data formats including a first data format. In some embodiments, a “second data format” can be used by a second instrument for communicating. The second data format may be different from a first data format. The second instrument may use a different data format from the first data format because, for example, the second instrument is manufactured by a different manufacturer than the other instruments within the subsystem. The second data format may specify the type, order, length, and structure of data messages, including header information, used by the second instrument. In some embodiments, the second data format may be a proprietary data format developed by the manufacturer of the second instrument.

A “subsystem” may include part of a complete system. In some embodiments, a subsystem may comprise a transport assembly, a controller, and one or more instruments used to process biological and/or chemical samples and that each communicate using the first data format.

A “transport assembly” may comprise any suitable hardware that can be used to transport a sample container. Exemplary transport assemblies may comprise one or more of pucks, conveyors, tracks, belts, grippers, and so forth.

A “translation module” may include software that can translate data from one format to another. For example, a translation module may receive data from one or more second instruments and translate the data into the first data format for use within the completed sample processing system. The translation module may also receive data from components (e.g., the controller, the transport assembly, and so forth) that use the first data format and translate the data into the second data format for use by the second instrument.

In the following description, for the purposes of explanation, specific details are set forth in order to provide a thorough understanding of embodiments of the present disclosure. However, it will be apparent that various embodiments may be practiced without these specific details. The figures and description are not intended to be restrictive.

Systems depicted in some of the figures may be provided in various configurations. Optionally, the systems may be configured as a distributed system where one or more components of the system are distributed across one or more networks in a cloud computing system. All features of the described systems are applicable to the described methods mutatis mutandis, and vice versa.

FIG. 1 illustrates a simplified block diagram of a completed sample processing system 100. The completed sample processing system 100 may include a transport assembly 105, a refrigeration unit 110, one or more instruments 115, one or more connectors 120, a controller 125, an adaptive connector 130, an adapter device 135, and an adapted instrument 140. In some embodiments, completed sample processing system 100 may include additional or fewer components. For example, in some embodiments, there may be more or fewer instruments 115. As another example, in some embodiments, completed sample processing system 100 may include a biohazard unit and/or a waste disposal unit.

The transport assembly 105 may be any suitable component for transporting test tubes (i.e., sample containers) throughout completed sample processing system 100. For example, transport assembly 105 may be a conveyor, a puck, a track, a gripper, and so forth. Transport assembly may communicate with controller 125 using the first data format. The first data format may specify the type, order, length, and structure of data messages. Upon receiving instructions from the controller 125, the transport assembly 105 may transport one or more test tubes to a desired location. For example, a test tube that has completed testing in instrument 115a may be moved by transport assembly 105 to the refrigeration unit 110.

The refrigeration unit 110 may be any suitable storage unit that maintains a specified temperature for storing the test tubes containing the biological and/or chemical samples. While a single refrigeration unit 110 is depicted, more than one refrigeration unit 110 may be in completed sample processing system 100. While named refrigeration unit 110, suggesting the specified temperature is cold, the refrigeration unit 110 may store the test tubes at any suitable temperature specified within the completed sample processing system 100. Refrigeration unit 110 may be physically coupled to the transport assembly 105 and communicatively coupled to the transport assembly 105 and/or controller 125.

Instruments 115 may be any machine or equipment that performs a specified function or test. Instruments 115 may be physically coupled to transport assembly 105 via connectors 120. As shown in FIG. 1, each instrument 115 may have an associated connector 120. Instruments 115 may be communicatively coupled to transport assembly 105 and/or controller 125. In some embodiments, connector 120 may be a wireless connector rather than a physical connector. Instruments 115 may communicate with transport assembly 105 and/or controller 125 using the first data format.

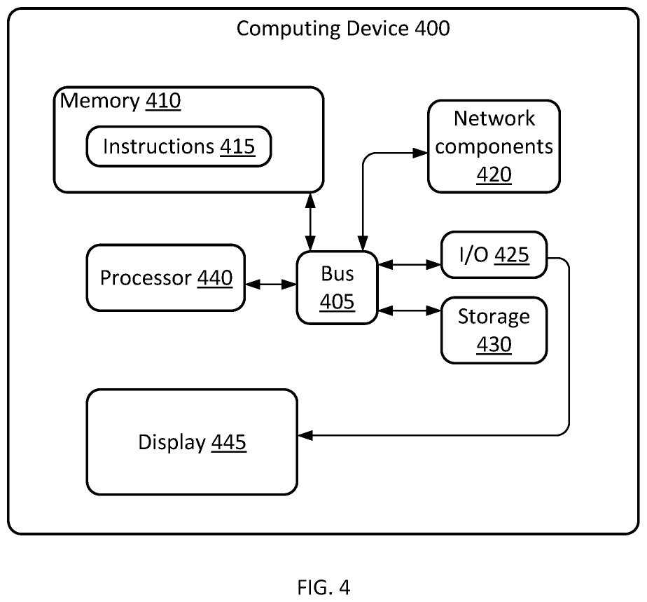

Controller 125 may be any suitable computer device, such as computer device 400 of FIG. 4, for controlling the components of completed sample processing system 100. Controller 125 may communicate with transport assembly 105, refrigeration unit 110, and instruments 115 using the first data format. Controller 125 may include a translation module as described in more detail with respect to FIG. 2. The controller 125 may use the translation module to translate data from the first data format to the second data format before transmitting the data to the adapted instrument 140. Controller 125 may include software modules, including the translation module, that controls the location and tests performed on each test tube in the completed sample processing system 110.

Adaptive connector 130 may be a mechanical and/or electrical connector that couples an adapter device 135 to transport assembly 105. In some embodiments, the adaptive connector 130 may be a hardware coupling device for coupling the adapter device 135 to the transport assembly 105. In some embodiments, the adaptive connector 130 may not be a separate physical component from adapter device 135. In some embodiments, the adapter device 135 may be wirelessly and communicatively coupled to the transport assembly 105 such that adaptive connector 130 is a wireless connection.

Adapter device 135 may be any suitable hardware and/or software component for operationally coupling the adapted instrument 140 to the transport assembly 105 and communicatively coupling the adapted instrument 140 to controller 125. The adapter device 135 may be a physical connection device having a physical connector for which the adapted instrument 140 may have a corresponding connector. In some embodiments, the manufacturer of the adapted instrument 140 may be different than the manufacturer of the transport assembly 105. In some embodiments, the manufacturer of the transport assembly 105 and the adapter 135 may use a proprietary data format (i.e., the first data format) that is different from a proprietary data format (i.e., the second data format) used by the adapted instrument 140. In some embodiments, adapter device 135 and adaptive connector 130 may not be separate and may instead be a wireless adapter 135 for wirelessly coupling the adapted instrument 140 to the transport assembly 105 and communicatively coupling the adapted instrument 140 to the controller 125 and the transport assembly 105.

In use, the transport assembly 105, refrigeration unit 110, instruments 115 coupled to transport assembly 105 using connectors 120, and controller 125, without the adapted instrument 140, may be a subsystem that uses a first data format for communication. It may be desirable to incorporate the adapted instrument 140 into the subsystem to create a completed sample processing system 100. The adaptive connector 130 may be coupled to the transport assembly 105 and the adapter device 135 may be coupled to the adaptive connector 130. In some embodiments, the adaptive connector 130 and the adapter device 135 may be a single component. The adapted instrument 140 may be coupled to the adapter device 135.

The adapted instrument 140 may be coupled to the adapter device 135 using a physical connection. In some embodiments, the physical connection can include male/female connectors and their connection can be detected by the adapter device 135 and/or the adapted instrument 140. Upon coupling the adapted instrument 140 to the adapter device 135, a plug-and-play process may begin. For example, the controller 125 may detect the adapted instrument 140 because, for example, the adapter device 135 may transmit a message to the controller 125 using the first data format indicating that the adapted instrument 140 has been coupled to the completed sample processing system 100. The second data format information may be provided to the controller 125, which may implement a translation module for translating data messages to and from the adapted instrument 140. In some embodiments, the translation module may be in the adapter device 135. As an example, of how the plug-and-play process may operate, the adapted instrument 140 may be plugged into adapter device 135. Adapter device 135 may receive a signal from adapted instrument 140. Adapter device 135 may not recognize adapted instrument 140, and may then send a notification to controller 125 that a new instrument has been added. The notification may include all or a portion of data from the signal from adapted instrument 140. In some embodiments, adapter device 135 may be able to identify the manufacturer of adapted instrument 140 from the signal and include that information in the notification to the controller 125. Using the notification and/or the data from the signal, controller 125 may identify the type of instrument and/or the manufacturer of adapted instrument 140. Using that information, the controller may have a lookup table, a link, or some other source from which controller 135 may obtain conversion data tables that may be used to translate instructions from the second data format used by adapted instrument 140 to the first data format. In some embodiments, a driver installation on controller 125 may launch in response to identification of the manufacturer and/or the model number of adapted instrument 140. The driver may provide the data tables and other information used to allow controller 125 to communicate with adapted instrument 140. In some embodiments, adapter device 135 may identify or locate the data tables and provide that information to controller 125. In some embodiments, adapted device 140 may provide the data tables to the adapter device 135, which may then send the data tables to controller 125. In some embodiments, data tables may not be used for translation. In such embodiments, any suitable information used to identify adapted instrument 140 and convert data between adapted instrument 140 and controller 125 may be used and/or shared during the plug-and-play process.

In some embodiments, adapted instrument 140 may be coupled to the adapter device 135 using a wireless connection. For example, Bluetooth communication may be used such that the adapter device 135 may scan for instruments and identify the adapted instrument 140 for coupling. Once the adapted instrument 140 is paired (i.e., coupled) to the adapter device 135, the plug-and-play process may begin.

As described above, attaching the adapted instrument 140 to the completed sample processing system 100 using the plug-and-play process segregates the components of the completed sample processing system 100 sufficiently such that when a new instrument is incorporated into the completed sample processing system 100, the entire completed sample processing system 100 need not go through regulatory approval. Rather, just the newly added instrument needs regulatory approval. As such, the cost and time associated with obtaining the regulatory approval is limited to a single instrument rather than the entire completed sample processing system 100.

FIG. 2 illustrates a simplified block diagram of communication components of a completed sample processing system 100. The completed sample processing system 100 may include transport assembly 105, refrigeration unit 110, instruments 115, controller 125, and adapted instrument 140. These components are as described with respect to FIG. 1.

Controller 125 may include controller interface 205, translation module 210, test and control software 215, and adapted instrument component 220. Controller interface 205 may be a communication interface or transceiver for transmitting data to and receiving data from transport assembly 105, refrigeration unit 110, and instruments 115. The controller interface 205 uses the first data format for processing the data messages flowing between the components.

Translation module 210 may be a software module that, when used in conjunction with one or more processors, may receive data messages from the adapted instrument component 220. The data messages from adapted instrument component 220 may be in a second data format that is different from the first data format. Adapted instrument component 220 may have a device identifier for the adapted instrument 140 to which it is associated. The identifier may be instrument specific or specific to a specific type of data format. The translation module 210 may convert the data messages from the first data format to the second data format, and vice versa. In some embodiments, the translation module 210 may include data tables which may map different conversion protocols or code for converting data between the second data format, and a first data format and other data formats. When the translation module 210 receives a data message, it may determine the destination of the data message and make the appropriate conversion based upon a received device identifier. The translation module 210 may be preprogrammed to otherwise recognize communication formats coming from the adapted instrument 140, and identify the conversion protocol needed to translate the data from the first data format to the second data format from the one or more data tables that may be maintained by the translation module. For example, machine learning may be used to identify synonyms and other like information from existing, known translation models. When a new message arrives from an instrument newly coupled to the system, machine learning may be used to parse the messages, identify the format of the message, and translate the message using a data message model, synonyms, similar protocols, and so forth. In some embodiments, there may be more than one instrument using differing data formats for messages. For example, a third instrument may also be coupled to the completed sample processing system 100 that uses a third data format different from the first data format and different from the second data format. The translation module 210 may detect the data format of the incoming data message and determine the destination of the data message and using that information make the appropriate conversion. If, for example, the data message comes from the third instrument and is intended for the test and control software 215, the translation module 210 may detect that the message is in the third data format and convert it to the first data format used by the controller 125. In some embodiments, the translation module 210 may be on the adapter device 135 rather than the controller 125. In such embodiments, the messages from the adapted instrument may be translated by the translation module 210 on the adapter device 135 and transmitted to the controller interface 205 of the controller 125.

Test and control software 215 may be the software application that is used by controller 125 to monitor and control where the sample containers (e.g., test tubes) within the completed sample processing system 100 are located as well as transmit instructions to the instruments 115, adapted instrument 140, and transport assembly 105 for moving sample containers from one instrument or location to another instrument or location, and for processing the various sample containers in the various instruments. The test and control software 215 may transmit data messages in the first data format and process data messages received in the first data format.

Adapted instrument component 220 may be a software module that is installed in controller 125 when the adapted instrument 140 is coupled to the adapter device 135. For example, the plug-and-play process may prompt installation of the adapted instrument component 220 onto controller 125. When a data message is transmitted from the adapted instrument 140 to the controller 135, the adapted instrument component 220 may receive the data message and send the data message to the translation module 210 for translation from the second data format to the first data format. Similarly, when the test and control software 215 generates a data message intended for the adapted instrument 140, the translation module 210 may translate the data message from the first data format to the second data format.

In use, test and control software 215 may generate a first data message indicating that adapted instrument 140 should receive a test tube, for example. The first data message may be in the first data format. Test and control software 215 may transmit the first data message to the translation module 210. Translation module 210 may convert the first data message from the first data format to the second data format. Once translated, translation module 210 may transmit the translated first data message to the adapted instrument component 220, which may transmit the translated first data message to the adapted instrument 140. The adapted instrument 140 may receive the test tube and may generate a second data message in the second data format indicating that it received the test tube. The adapted instrument 140 may transmit the second data message to the controller 125 indicating the location of a test tube. The controller 135 may receive the second data message at the adapted instrument component 220. The adapted instrument component 220 may transmit the second data message to the translation module 210. The translation module 210 may convert the second data message from the second data format to the first data format and transmit the translated second data message to the test and control software 215. In this way, the controller 125 may communicate with and control the adapted instrument 140 when adapted instrument 140 uses a different data format than the controller 125 uses.

In use, the transport assembly 105, instruments 115, and refrigeration unit 110 may communicate in a first data format with controller 125. Data messages in the first data format may be received by the controller interface 205 and transmitted to the test and control software 215 for processing and response. Data messages from the test and control software 215 intended for any of the transport assembly 105, the instruments 115, or the refrigeration unit 110 may be transmitted in the first data format using the controller interface 205.

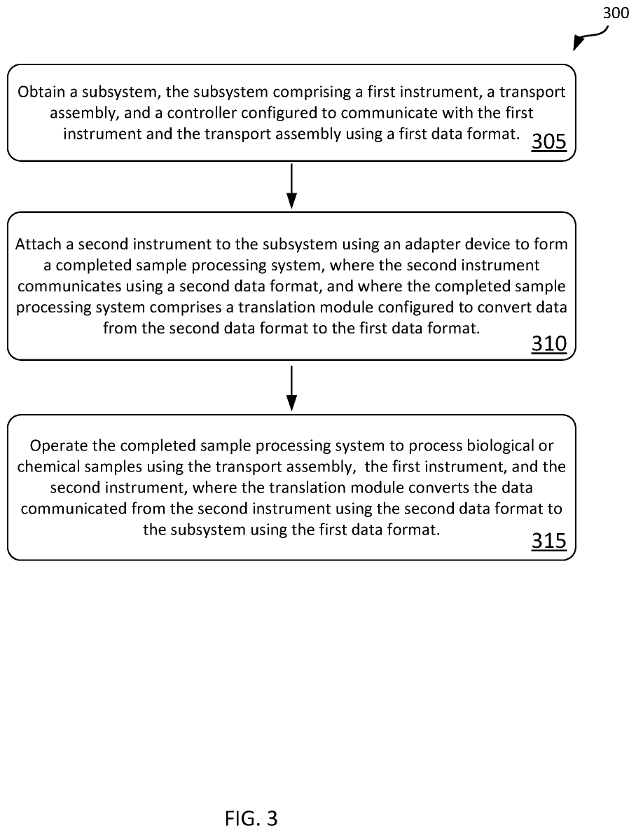

FIG. 3 illustrates a sample method 300 for incorporating an instrument that uses a different data format into a completed sample processing system that uses a first data format.

At block 305, the method 300 may begin with obtaining a subsystem. The subsystem may include a first instrument, such as instrument 115. The subsystem may include a transport assembly, such as transport assembly 105. The subsystem may include a controller, such as controller 125. The subsystem may use a first data format. For example, the controller, instrument, and transport assembly may communicate using the first data format.

At block 310, the method may continue with attaching a second instrument to the subsystem using an adapter device to form a completed sample processing system. The second instrument may be, for example, the adapted instrument 140. The second instrument may be attached to the subsystem using an adapter device, such as adapter device 135. After attaching the second instrument, a completed sample processing system is formed such as completed sample processing system 100. The second instrument may communicate using a second data format. The completed sample processing system may include a translation module, such as translation module 210. The translation module may be configured to convert data from the second data format to the first data format. The translation module may also be configured to convert data from the first data format to the second data format.

At block 315, the method may continue with operating the completed sample processing system to process biological or chemical samples using the transport assembly, the first instrument, and the second instrument. For example, test tubes containing biological or chemical samples may be transported between instruments and/or locations using the transport assembly. The instruments may perform tests and other procedures on the biological or chemical samples. When the second instrument using the second data format communicates with the controller, the translation module may convert the data messages from the second data format to the first data format. Further, when the controller communicates with the second instrument, the translation module may convert the data messages from the first data format to the second data format.

FIG. 4 illustrates a block diagram of an example computing device 400. Computing device 400 can be any of the described computers herein including, for example, controller 125. The computing device 400 can be or include, for example, a laptop computer, desktop computer, tablet, e-reader, smart phone or mobile device, smart watch, personal data assistant (PDA), or other electronic device.

The computing device 400 can include a processor 440 interfaced with other hardware via a bus 405. A memory 410, which can include any suitable tangible (and non-transitory) computer readable medium, such as RAM, ROM, EEPROM, or the like, can embody program components (e.g., instructions 415) that configure operation of the computing device 400. In some examples, the computing device 400 can include input/output (“I/O”) interface components 425 (e.g., for interfacing with a display 445, keyboard, or mouse) and additional storage 430.

The computing device 400 can include network components 420. Network components 420 can represent one or more of any components that facilitate a network connection. In some examples, the network components 420 can facilitate a wireless connection and include wireless interfaces such as IEEE 802.11, Bluetooth, or radio interfaces for accessing cellular telephone networks (e.g., a transceiver/antenna for accessing CDMA, GSM, UMTS, or other mobile communications network). In other examples, the network components 420 can be wired and can include interfaces such as Ethernet, USB, or IEEE 1394.

Although FIG. 4 depicts a single computing device 400 with a single processor 440, the system can include any number of computing devices 400 and any number of processors 440. For example, multiple computing devices 400 or multiple processors 440 can be distributed over a wired or wireless network (e.g., a Wide Area Network, Local Area Network, or the Internet). The multiple computing devices 400 or multiple processors 440 can perform any of the steps of the present disclosure individually or in coordination with one another.

Embodiments of the invention provide for a number of advantages. By using an adapter device, embodiments of the invention can automatically allow different instruments that may operate using different data formats to operate within a completed laboratory automation system. It is not necessary to manually hard code and customize a particular instrument to be added to a subsystem. This results in a significant time savings and is more convenient for any entity that wishes to operate a completed laboratory automation system. Further, regulatory approval is minimized. A subsystem that has previously been approved is not required to undergo approval a second time with the additional instruments. Rather, the additional instruments may be approved individually, saving time and money, and can be added in this plug-and-play fashion to result in a completed sample processing system that has regulatory approval with minimized time and cost.

Each of the calculations or operations described herein may be performed using a computer or other processor having hardware, software, and/or firmware. The various method steps may be performed by modules, and the modules may comprise any of a wide variety of digital and/or analog data processing hardware and/or software arranged to perform the method steps described herein. The modules optionally comprising data processing hardware adapted to perform one or more of these steps by having appropriate machine programming code associated therewith, the modules for two or more steps (or portions of two or more steps) being integrated into a single processor board or separated into different processor boards in any of a wide variety of integrated and/or distributed processing architectures. These methods and systems will often employ a tangible media embodying machine-readable code with instructions for performing the method steps described above. Suitable tangible media may comprise a memory (including a volatile memory and/or a non-volatile memory), a storage media (such as a magnetic recording on a floppy disk, a hard disk, a tape, or the like; on an optical memory such as a CD, a CD-R/W, a CD-ROM, a DVD, or the like; or any other digital or analog storage media), or the like.

Different arrangements of the components depicted in the drawings or described above, as well as components and steps not shown or described are possible. Similarly, some features and sub-combinations are useful and may be employed without reference to other features and sub-combinations. Embodiments of the present disclosure have been described for illustrative and not restrictive purposes, and alternative embodiments will become apparent to readers of this patent. In certain cases, method steps or operations may be performed or executed in differing order, or operations may be added, deleted or modified. It can be appreciated that, in certain aspects of the present disclosure, a single component may be replaced by multiple components, and multiple components may be replaced by a single component, to provide an element or structure or to perform a given function or functions. Except where such substitution would not be operative to practice certain embodiments of the present disclosure, such substitution is considered within the scope of the present disclosure.

It is to be understood that the figures and descriptions of embodiments of the present disclosure have been simplified to illustrate elements that are relevant for a clear understanding of the present disclosure. Those of ordinary skill in the art will recognize, however, that these and other elements may be desirable. However, because such elements are well known in the art, and because they do not facilitate a better understanding of the present disclosure, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the present disclosure, a single component may be replaced by multiple components, and multiple components may be replaced by a single component, to provide an element or structure or to perform a given function or functions. Except where such substitution would not be operative to practice certain embodiments of the present disclosure, such substitution is considered within the scope of the present disclosure. A recitation of “a”, “an” or “the” is intended to mean “one or more” unless specifically indicated to the contrary.

The examples presented herein are intended to illustrate potential and specific implementations of the present disclosure. It can be appreciated that the examples are intended primarily for purposes of illustration of the present disclosure for those skilled in the art. There may be variations to these diagrams or the operations described herein without departing from the spirit of the present disclosure. For instance, in certain cases, method steps or operations may be performed or executed in differing order, or operations may be added, deleted or modified.

Furthermore, whereas particular embodiments of the present disclosure have been described herein for the purpose of illustrating the present disclosure and not for the purpose of limiting the same, it will be appreciated by those of ordinary skill in the art that numerous variations of the details, materials and arrangement of elements, steps, structures, and/or parts may be made within the principle and scope of the present disclosure without departing from the present disclosure as described in the claims.

What is claimed is:

1. A method, comprising:
attaching a second instrument to a subsystem using an adapter device to form a completed sample processing system, wherein:
the subsystem comprises a first instrument, a transport assembly, and a controller configured to communicate with the first instrument and the transport assembly using a first data format,
the second instrument communicates using a second data format, and
the completed sample processing system comprises a translation module configured to convert data from the second data format to the first data format; and
operating the completed sample processing system to process biological or chemical samples using the transport assembly, the first instrument, and the second instrument, wherein the translation module converts the data communicated from the second instrument using the second data format to the subsystem using the first data format.

2. The method of claim 1, wherein the adapter device is a wireless connection device that facilitates wireless communication between the second instrument and the transport assembly.

3. The method of claim 1, wherein the adapter device comprises a physical connection port for physically coupling the second instrument to the transport assembly.

4. The method of claim 1, wherein the translation module is installed in the controller.

5. The method of claim 1, wherein the translation module is installed in the adapter device.

6. A subsystem, comprising:
a transport assembly;
a first instrument;
an adapter device configured to couple the transport assembly and a second instrument;
a controller configured to communicate with the first instrument and the transport assembly using data in a first data format; and
a translation module configured to convert data communicated by the second instrument in a second data format to the first data format for receipt by the transport assembly.

7. The subsystem of claim 6, wherein the adapter device is a wireless connection device that facilitates wireless communication between the second instrument and the transport assembly.

8. The subsystem of claim 6, wherein the adapter device comprises a physical connection port for physically coupling the second instrument to the transport assembly.

9. The subsystem of claim 6, wherein the translation module is installed in the controller.

10. The subsystem of claim 6, wherein the translation module is installed in the adapter device.

11. A system, comprising:
a first instrument,
a transport assembly,
a controller configured to communicate with the first instrument and the transport assembly using a first data format; a second instrument coupled to the transport assembly with an adapter device, wherein the second instrument communicates using a second data format; and
a translation module configured to convert data communicated between the second instrument and the controller.

12. The system of claim 11, wherein the translation module converts data from the second instrument in the second data format to the first data format for receipt by the controller and the translation module converts data from the controller in the first data format to the second data format for receipt by the second instrument.

13. A computer-readable memory device having stored thereon instructions that, when executed by one or more processors, cause the one or more processors to:
receive first data from a first component of a plurality of components of a sample processing subsystem, the plurality of components comprising a transport assembly and a first instrument, the first data in a first data format, and the first data for transmission to a second instrument that communicates using a second data format;
determining the second data format;
converting the first data from the first data format to the second data format to generate second data; and
transmitting the second data to the second instrument.
